(19) United States
(12) Patent Application Publication
(10) Pub. No.: US 2014/0371322 A1
(43) Pub. Date: Dec. 18, 2014

(54) Phenoxybenzamine Transdermal Composition

(71) Applicant: Andrew B. Glasnapp, Sugar Land, TX (US)

(72) Inventor: Andrew B. Glasnapp, Sugar Land, TX (US)

(73) Assignee: PROFESSIONAL COMPOUNDING CENTERS OF AMERICA, Houston, TX (US)

(21) Appl. No.: 13/917,292

(22) Filed: Jun. 13, 2013

(51) Int. Cl. A61K 31/138

(52) U.S. Cl. CPC A61K 31/138
USPC 514/652

(57) ABSTRACT

A phenoxybenzamine transdermal composition for treating neuropathic pain is disclosed. The phenoxybenzamine transdermal composition may include phenoxybenzamine in a concentration of about 5 mg/g to about 120 mg/g, with about 15 mg/g being preferred, in combination with a pharmaceutically suitable permeation enhancer that may be included in amounts of about 20% by weight to about 99.95% by weight, with about 50% by weight being preferred. Permeation enhancer composition within disclosed phenoxybenzamine transdermal composition may improve penetration of phenoxybenzamine in a patient's tissue or skin. The phenoxybenzamine transdermal composition may provide a long duration blockade of sensitized pain receptors of 24 hours or more, resulting in an effective treatment for neuropathic pain with lower concentrations of phenoxybenzamine and requiring fewer applications.

### CROSS-REFERENCE TO RELATED APPLICATIONS

N/A

### BACKGROUND

1. Field of the Disclosure

The present disclosure relates in general to pharmaceutical compositions, and more specifically to transdermal compositions including phenoxybenzamine for treatment of neuropathic pain.

2. Background Information

Pain is typically experienced when the free nerve endings of pain receptors are subject to mechanical, thermal, chemical, or other noxious stimuli. When treating pain, it is important to understand the distinction between acute and persistent or chronic pain. Acute pain occurs as a result of tissue injury, and is mediated by chemical, mechanical, or thermal stimulation of pain receptors known as nociceptors. On the other hand, chronic or persistent pain is a disease that serves no protective biological function, and may predominantly constitute chronic inflammatory pain (e.g. arthritis) or “neuropathic pain”. To date, most drug discovery approaches for neuropathic pain have been based on symptom management, directed at the most commonly described clinical symptoms namely spontaneous pain, mechanical and cold allodynia, hyperalgesia, and hyperpathia.

Some types of neuropathic pain disorders involve myofascial trigger points, which are hyperirritable spots in skeletal muscle associate with palpable nodules in taut bands of muscle fibers. These myofascial trigger points may induce a characteristic pattern of pain, tingling, or numbness in response to sustained pressure, as well as a local twitch of the taut band when the myofascial trigger point is distorted transversely. Although the taut band may be several centimeters long, a myofascial trigger point may measure only a few millimeters in diameter. Myofascial pain may include referred pain, referred tenderness, or referred autonomic phenomena, such as vasoconstriction, coldness, sweating, pilomotor response, ptosis, and hypersecretion.

Although there are numerous available therapies for acute pain caused by stimulation of the nociceptors, especially treatment with opioid and non-steroidal anti-inflammatory drugs (NSAIDs), or even inserting needles in myofascial trigger points for myofascial pain, neuropathic pain has not yet found effective enough treatments. Since nociception is not important for chronic neuropathic pain, analgesics only play a minor role. Drugs used to treat neuropathic pain may be broadly categorized into agents interacting with the ascending neurons like sodium and calcium channels blockers, N-methyl D-aspartate (NMDA) and neurokinin-1 (NK-1) receptor antagonists, or drugs that enhance descending inhibitory fiber activity, such as tricyclic antidepressants (TCA's). Nevertheless, these agents are associated with significant side effect profiles and these drugs have been found effective more by chance than by clinical trials.

Administration of phenoxybenzamine, a non-selective, irreversible alpha antagonist, has been reported for certain neuropathic diseases such as reflex sympathetic dystrophy/complex regional pain syndromes (RSD/CRPS) using an intravenous regional block of an affected body part or by oral administration. Intravenous (injectable) and oral administration of phenoxybenzamine have also been employed for treating high blood pressure. While treatments with phenoxybenzamine are considerably beneficial, postural hypotension is a prominent side effect, along with disorientation and ejaculatory problems.

For the foregoing reasons, it would be beneficial to develop new methods and compositions for treating neuropathic pain that may be safe, efficacious, well-tolerated, and which may be administered more conveniently and over an extended period of time without harsh side effects.

### SUMMARY

The present disclosure describes a phenoxybenzamine transdermal composition that may be used to treat neuropathic pain. Phenoxybenzamine transdermal composition may include phenoxybenzamine in a concentration of about 5 mg/g to about 120 mg/g, with about 15 mg/g being preferred, in combination with a pharmaceutically suitable permeation enhancer that may be included in amounts of about 20% by weight to about 99.95% by weight, with about 50% by weight being preferred. Phenoxybenzamine transdermal composition may be administered in a suitable dosage form, such as a gel.

Phenoxybenzamine is the only non-competitive alpha blocker known to date, providing a long duration blockade of alpha adrenergic receptors that may last for about 24 hours or more, and thus of sensitized pain pathways, resulting in relief of neuropathic pain in a subject. For example, diseases related to neuropathic pain, such as myofascial pain from myofascial trigger points, may be efficiently treated using disclosed phenoxybenzamine transdermal composition, applying the latter on myofascial trigger points.

Permeation enhancer compositions may be added at a suitable concentration to phenoxybenzamine transdermal composition, increasing permeation power and hence accelerating onset phenoxybenzamine transdermal composition's effect. Permeation enhancer composition included in phenoxybenzamine transdermal composition may be a liquid or semi-liquid that includes phospholipids. Permeation enhancer compositions may include one or more naturally occurring substances, including one or more phospholipids, one or more oils rich in essential fatty acids (behenic acid, and oleic acid), one or more skin lipids, and one or more butters rich in linoleic acid and linolenic acid. The ingredients within permeation enhancer composition may act synergistically to increase the skin permeation to water and oil soluble products.

Benefits from administering disclosed phenoxybenzamine composition transdermally may include that this administration route is not invasive, may be self-administered by the patient, and represents an efficient route for rapid and complete plasma delivery. The long residence time of phenoxybenzamine may reduce the concentration and the number of applications of phenoxybenzamine transdermal composition to the subject, and thus the side effects of phenoxybenzamine. Furthermore, rapid onset of effect facilitates the opportunity for the physician and/or the patient to more effectively titrate the dose needed for an adequate therapeutic effect. Types of neuropathic pain that may be treated with phenoxybenzamine transdermal composition may include fibromyalgia, myofascial pain syndrome, tension headache, temporomandibular joint dysfunction (TMD), neck and low back pain syndromes, migraine headache, sciatica, plantar fasciitis, complex regional pain syndrome, and restless leg syndrome, among others.

Numerous other aspects, features and benefits of the present disclosure may be made apparent from the following detailed description taken together with the drawing figures.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIGS. 1A-B shows the two types of alpha adrenergic antagonism, according to prior art.

FIG. 2 shows application of phenoxybenzamine transdermal composition on myofascial trigger points, according to an embodiment.

### DETAILED DESCRIPTION

The present disclosure is here described in detail with reference to embodiments illustrated in the drawings, which form a part hereof. Other embodiments may be used and/or other changes may be made without departing from the spirit or scope of the present disclosure. The illustrative embodiments described in the detailed description are not meant to be limiting of the subject matter presented here.

### DEFINITIONS

As used here, the following terms have the following definitions:

“Treat”, “Treating”, and “Treatment” refer to reduction in severity and/or frequency of symptoms, elimination of symptoms and/or underlying cause, prevention of the occurrence of symptoms and/or their underlying cause, and improvement or remediation of damage.

“Transdermal drug delivery” refers to administration of a drug to the skin surface of an individual so that the drug passes through the skin tissue and into the individual's blood stream, therefore providing a systemic effect. The term “transdermal” is intended to include “transmucosal” drug administration, for example, administration of a drug to the mucosal surface of an individual, such as: sublingual, buccal, vaginal, rectal, so that the drug passes through the mucosal tissue and into the individual's blood stream. Unless otherwise stated or implied, the terms “topical drug administration” and “transdermal drug administration” are used interchangeably.

“Alpha adrenergic receptor” refers to molecules on a surface of a cell or within the cell that, upon interaction with catecholamines, especially norepinephrine (noradrenaline) and epinephrine (adrenaline), control several physiological processes such as vasoconstriction, intestinal relaxation, and pupil dilation, among others.

“Alpha adrenergic antagonist” or “Alpha blocker” refers to a drug that opposes the excitatory effects of norepinephrine released from sympathetic nerve endings at alpha adrenergic receptors and that causes vasodilation and a decrease in blood pressure.

“Noncompetitive antagonism” refers to an action in which an alpha adrenergic antagonist removes an alpha adrenergic receptor or the alpha adrenergic receptor's response potential from a nervous system, preventing the alpha adrenergic antagonist from producing an effect at a receptor site by irreversible change to the receptor or to the receptor's capacity to respond. Noncompetetitive antagonism is not reversible by increasing concentration of an alpha adrenergic agonist.

“Permeation enhancement” refers to an increase in the permeability of the skin or mucosal tissue to the selected active pharmaceutical ingredient.

“Vehicle” refers to a substance of no therapeutic value that is used to convey an active medicine for administration.

“Phospholipids” refers to fat-like organic compounds that resemble triglycerides, but have a fatty acid with a phosphate polar group.

“Liposomes” refers to artificially prepared vesicles made of lipid bilayer, and have concentric phospholipid bilayers.

### DESCRIPTION OF THE DRAWINGS

The present disclosure describes a phenoxybenzamine transdermal composition that may include phenoxybenzamine combined with a suitable natural permeation enhancement (NPE) composition. This phenoxybenzamine transdermal composition may be used to treat, alleviate, prevent, diminish, or otherwise ameliorate symptoms associated with neuropathic pain in a subject. Application of phenoxybenzamine transdermal composition does not induce oversedation, and side effects of phenoxybenzamine may be diminished, if existent.

Neuropathic Pain

Neuropathic pain, a type of pain associated with disease or injury to the peripheral or central nervous system, is a common symptom of a heterogeneous group of conditions, including diabetic neuropathy, trigeminal neuralgia, postherpetic neuralgia, and spinal cord injury. There might also be a neuropathic component in the pain experienced by patients with cancer, degenerative diseases, or neurologic conditions that have so far gone unnoticed.

Table 1 shows the classification of neuropathic pain by etiology and anatomical localization.

TABLE 1
Classification of Neuropathic Pain

| Periphery | Spinal | Brain |
|---|---|---|
| Neuropathies | Multipe sclerosis | Syringobulbia |
| Traumatic nerve injury | Spinal injuries | Stroke |
| Plexus avulsion | Myelopathies | Multiple sclerosis |
| Amputation | Ischaemic lesions | Parkinson's disease |
| Neuralgia | Syringomyelia | |
| Compression | Chordotomy | |
| HIV infection | Cancer compression | |
| Polyradiculitis | | |

From the clinical point of view, neuropathic pain represents a heterogeneous group of etiologically different diseases ranging from cancer to diabetes. Neuropathic pain also differs with respect to location; disorders may exist anywhere between the peripheral receptor and the brain. Despite the heterogeneity in etiology and anatomical location, neuropathic pains share certain characteristics. Typically, patients with neuropathic pain complain of spontaneous pains (those that arise without detectable stimulation) and evoked pains (abnormal responses to stimuli). Spontaneous pains may be continuous, steady, and ongoing or paroxysmal, episodic, and intermittent.

The relationship between neuropathic pain symptoms and key cellular and molecular mechanisms are not yet fully understood. However, studies suggest that the major cellular mechanisms may include ectopic or spontaneous nerve activity and peripheral and central hyperexcitability, phenotypic changes in pain conducting pathways, secondary neurodegeneration, and morphological reorganization. It is also recognized that episodic inflammation, and chronic inflammatory conditions, may cause nerve injury, encouraging a broader appreciation of the heterogeneity of neuropathic pain etiology.

Studies have suggested that the sympathetic nervous system has a role in neuropathic pain. Preclinical models of neuropathic and inflammatory pain show up-regulation of alpha adrenergic receptors, alpha adrenergic receptor supersensitivity, and functional coupling between sympathetic efferent and sensory afferent fibers.

Alpha Adrenergic Receptors

The ends of some nerves release norepinephrine when the nerve is stimulated. This chemical then stimulates alpha adrenergic receptors. These receptors are tiny structures which occur on cells in various parts of the body including the heart, smooth muscle, and blood vessels. When these receptors are stimulated, they may cause various effects.

Alpha adrenergic receptors exist on peripheral sympathetic nerve terminals and are divided into two subtypes, alpha 1 and alpha 2. Sympathetic nerves are present at the adventitial-medial border of arteries and increase of norepinephrine at these sites causes constriction of the arteries.

Alpha 1 adrenergic receptors are found throughout the body as well as in the brain, in both central and peripheral nervous systems. Alpha 1 adrenergic receptors may also play critical roles elsewhere in controlling contraction and growth of smooth and cardiac muscle. In the Central Nervous System (CNS) alpha 1 adrenergic receptors are found mostly postsynaptically and have an excitatory function; peripherally they are responsible for contraction and are situated on vascular and on non-vascular smooth muscle. Alpha 1 adrenergic receptors on vascular smooth muscle are located intrasynaptically and function in response to neurotransmitter release. For non-vascular smooth muscle, alpha 1 adrenergic receptors may be found on the liver causing hepatic glycogenolysis and potassium release. On the heart, alpha 1 adrenergic receptors mediate a positive inotropic effect. Alpha 1 adrenergic receptors may as well cause relaxation of GI smooth muscle and decrease salivary secretion.

Alpha 2 adrenergic receptors are found in both the central and peripheral nervous system and serve to produce inhibitory functions. Alpha 2 adrenergic receptors bind both norepinephrine released by sympathetic postganglionic fibers and epinephrine released by the adrenal medulla, binding epinephrine with slightly higher affinity. Alpha 2 adrenergic receptors are generally located on vascular prejunctional terminals (presynaptic alpha 2 adrenergic receptors) where they inhibit the release of norepinephrine in a form of negative feedback. Alpha 2 adrenergic receptors are also located on vascular smooth muscle cells of certain blood vessels (postsynaptic alpha 2 adrenergic receptors), such as blood vessels found in skin arterioles or on veins, proximate to the more abundant alpha 1 adrenergic receptors. Activation of postsynaptic alpha 2 adrenergic receptors causes platelet aggregation and blood vessel constriction.

Alpha 2 adrenergic receptor agonists as well as alpha 1 adrenergic antagonists are generally used for treatment of hypertension.

Alpha Adrenergic Blockade

Alpha blockers work by blocking alpha adrenergic receptors, which may prevent these receptors to receive certain nerve impulses such as norepinephrine. This norepinephrine blockade usually results in vasodilation and a decrease in blood pressure. Other applications of alpha blockers, when applied at suitable concentrations, may involve sedation of the damaged nerves that may cause neuropathic pain.

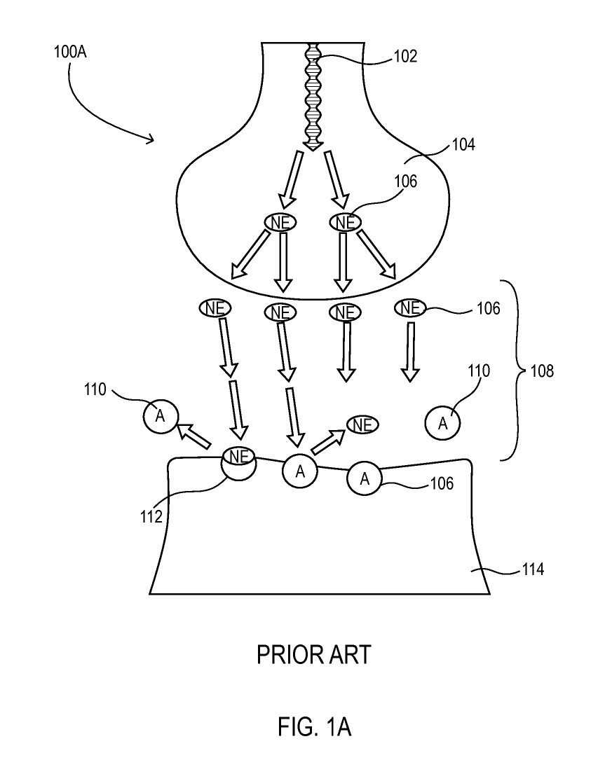

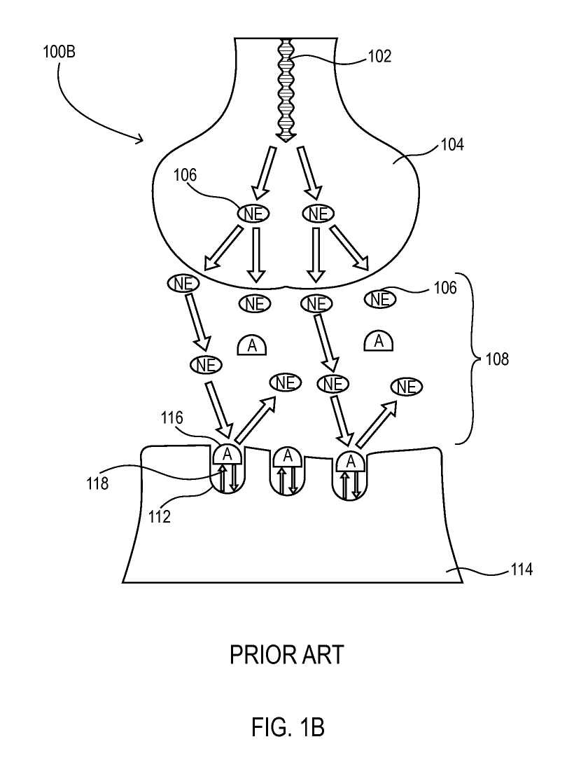

FIG. 1 shows the two types of alpha adrenergic antagonism. FIG. 1A depicts a competitive alpha antagonism 100A, while FIG. 1B depicts a non-competitive alpha antagonism 100B.

Competitive alpha antagonism 100A from FIG. 1A works when nerve impulses 102 from adrenergic nerve 104 release norepinephrine 106 (NE), which may then cross synaptic cleft 108 and reach competitive alpha blockers 110 (A) that block alpha adrenergic receptors 112 in effector cells 114. Effector cells 114 may include muscles, glands, and organs, among others. Effect of competitive alpha blockers 110 may be reduced by increasing norepinephrine 106 concentrations. Examples of competitive alpha blockers 110 may include phentolamine and tolazoline.

On the other hand, non-competitive alpha antagonism 100B, shown in FIG. 1B, works when nerve impulses 102 from adrenergic nerve 104 release norepinephrine 106, which may then cross synaptic cleft 108 and reach non-competitive alpha blockers 116 (A) that block alpha adrenergic receptors 112 in effector cells 114. Non-competitive alpha blockers 116 make a covalent bond 118 with alpha 1 and alpha 2 adrenergic receptors, providing a long duration blockade of alpha adrenergic receptors 112 that may not be reversed by increasing norepinephrine 106 concentration, unlike competitive alpha blockers 110. Non-competitive alpha blocker 116 may include phenoxybenzamine.

Alpha blockers are generally used in the treatment of several conditions, such as Raynaud's disease, hypertension, and scleroderma. Alpha blockers may also be used for treating anxiety and panic disorders, such as generalized anxiety disorder, panic disorder, or posttraumatic stress disorder (PTSD). While alpha blockers are commonly used to treat hypertension, they are also used to treat the symptoms of benign prostatic hyperplasia (BPH). Alpha blockers may additionally be used in the treatment of neuropathic pain.

Phenoxybenzamine Transdermal Composition

According to an embodiment, a phenoxybenzamine transdermal composition for treating neuropathic pain may include phenoxybenzamine in a dose of about 5 mg/g to about 120 mg/g, with about 15 mg/g being preferred, in combination with a pharmaceutically suitable NPE composition that may be included in concentrations of about 20% by weight to about 99.95% by weight, with about 50% by weight being preferred.

In one embodiment, disclosed phenoxybenzamine transdermal composition may be administered in gel form. In other embodiments, phenoxybenzamine transdermal composition may be administered in other suitable topical dosage forms such as an ointment, cream, gel, emulsion (lotion), oil, or similar formulation, employing suitable vehicles for each dosage form. Additionally, phenoxybenzamine transdermal composition may include customary excipient additives, such as vegetable oils including almond oil, olive oil, peach kernel oil, groundnut oil, castor oil and the like, animal oils, DMSO, fat and fat-like substances, lanolin lipoids, phosphatides, hydrocarbons such as paraffin, petroleum jelly, waxes, detergent emulsifying agents, lecithin, alcohols, carotin, glycerol, glycerol ethers, glycols, glycol ethers, polyethylene glycol, polypropylene glycol, non-volatile fatty alcohols, acids, esters, volatile alcoholic compounds, urea, talc, cellulose derivatives, and preservatives, among others.

Phenoxybenzamine

Phenoxybenzamine is the only non-competitive alpha blocker 116 known to date. Chemically, phenoxybenzamine is a weak base with a pka (dissociation constant) of 6.58 and an octanol/water log P partition coefficient of 4.6. Thus, phenoxybenzamine is highly lipophilic and may pass across cell membranes readily. Therefore, transdermal routes of administration may be efficient for the rapid and complete delivery of phenoxybenzamine to the plasma when combined with a suitable permeation enhancer, soothing neuropathic pain in the affected region in a fast and efficient manner. Furthermore, there may be a decrease of phenoxybenzamine concentration within phenoxybenzamine transdermal composition when compared to an oral dose, reducing risks of unwanted side effects. This may be a result of a long duration blockade of alpha adrenergic receptors 112 by phenoxybenzamine, reducing the number of applications to the subject and thus the side effects of phenoxybenzamine.

Phenoxybenzamine produces long-lasting insurmountable block of alpha adrenergic receptors 112 (from about 14 to about 48 hours) due to the covalent bond 118 that allows phenoxybenzamine bind to alpha adrenergic receptors 112, thereby preventing surges of blood pressure that may occur when large quantities of catecholamines (i.e. epinephrine, norepinephrine, and dopamine) are released from an affected adrenergic nerve 104. Phenoxybenzamine may additionally inhibit reuptake of released norepinephrine 106 by adrenergic nerves 104. Furthermore, phenoxybenzamine may block histamine (H₁), acetylcholine, and serotonin receptors, in addition to alpha adrenergic receptors 112. When circulating levels of catecholamines are low, phenoxybenzamine may produce a vasodilation relative to basal vessel tone due to blockade of an alpha adrenergic receptor 112. When an affected limb or body part suffers from neuropathic pain, a cold skin and other sensations may be the effect of a supersensitivity to catecholamines in the affected area, which, when blocked by phenoxybenzamine, may result in a sedation of effector cells 114 proximate to the area of application, and therefore a diminished neuropathic pain sensation.

Action of phenoxybenzamine in non-competitive antagonism of calmodulin may also be considered a relevant aspect of the mechanism of action of phenoxybenzamine. Calmodulin is a calcium-binding messenger protein expressed in eucaryotic cells that transduces calcium signals by binding calcium ions and then modifying interactions with various target proteins. Calmodulin mediates many crucial processes, including inflammation, metabolism, apoptosis, smooth muscle contraction, intracellular movement, short-term and log-term memory, and immune response.

In association with depolarization of the presynaptic nerve ending (adrenergic nerve 104), calcium (Ca++) enters the nerve terminal from adrenergic nerve 104, combines with calmodulin, which then activates a Ca++/calmodulin-dependent protein kinase; this kinase then phosphorylates synapsin I, which mediates the mobilization of vesicles containing neuromediator, allowing for their release into synaptic cleft 108. This process is believed to operate in release of glutamate, and in central sensitization to pain mediated via action of glutamate on N-methyl-D-aspartate (NMD A) receptors. In central neurons, Ca++/calmodulin-dependent protein kinase may be autophosphorylated to a form that may no longer be dependent upon Ca++ to maintain its active state, resulting in a persistence of its effects, such as sustained glutamate release, and may thus contribute to amplification of pain perception in certain syndromes.

Because of a non-competitive mechanism of antagonism of alpha adrenergic receptors 112, phenoxybenzamine does not have a true half time of elimination, providing a long duration block of sensitized pain pathways. The half time of the action of phenoxybenzamine may be best described as the half time of re-synthesis of alpha adrenergic receptors 112, or the slow spontaneous hydrolytic cleavage of covalent bond 118, which may be a matter of days. The half time of re-synthesis of calmodulin is another factor that may determine the duration of action of phenoxybenzamine.

Many of the adverse effects of phenoxybenzamine derive from alpha adrenergic receptor blockade. The most important side effects are postural hypotension and tachycardia, although other side effects may include nasal congestion, miosis, and inhibition of ejaculation, among others. Less common effects may include confusion, drowsiness, dryness of mouth, fatigue, headache, and gastrointestinal irritation. These side effects may be regulated by adjusting the dose of phenoxybenzamine. Therefore, due to a reduced dose of phenoxybenzamine because fewer applications may be required to soothe neuropathic pain, side effects may be substantially reduced.

Natural Permeation Enhancement (NPE) Composition

NPE composition within phenoxybenzamine transdermal composition may allow physicians to prescribe increased concentrations of multiple APIs to be administered transdermally, permitting a reduction of the amount of applied phenoxybenzamine transdermal composition needed by patients to treat neuropathic pain and increasing permeation percentages and effects of each API. Because NPE composition may allow transdermal application of phenoxybenzamine transdermal composition, there may be no effect on the liver and other parts of the digestive system and there may also be a reduction of systemic concentrations, leading to a decrease of adverse side effects.

NPE composition may include one or more naturally occurring substances, including one or more phospholipids, one or more oils rich in essential fatty acids, behenic acid, and oleic acid, one or more skin lipids, and one or more butters rich in linoleic acid and linolenic acid. According to an embodiment, NPE composition may be employed as a penetration enhancer for a number of different compounds, including topical cosmetics and pharmaceutical formulations. While NPE composition may be safe and effective, this composition may include natural ingredients which may assist with penetration of APIs through the skin. NPE composition having fatty acid micro-particles described here may include, among other components, behenic acid, oleic acid, omega-3 fatty acids, and phospholipids. The use of a NPE composition may eliminate need for pre-encapsulation of APIs.

As mentioned, NPE composition described here may include one or more naturally occurring substances, including one or more phospholipids, one or more oils rich in essential fatty acids, behenic acid, and oleic acid, one or more skin lipids, and one or more butters rich in linoleic acid and linolenic acid. The ingredients within NPE composition may act synergistically to increase the skin permeation of water and oil soluble products. NPE composition, which is a solution, may be added to a gel or emulsion at a given percent to give permeation power to the phenoxybenzamine transdermal composition. When NPE composition is prepared, liposomes may be formed from the fatty acids, including behenic acid and oleic acid that may be present in the one or more oils, and may be stabilized by the phospholipids in the composition. More specifically, when NPE composition is added to water or a water-incorporating composition, liposomes may be formed.

In some embodiments, liposomes may be filled with drugs or other APIs and may be used to deliver these drugs. Liposomes may include naturally-derived phospholipids with mixed lipid chains or other surfactants. In some embodiments, the liposomes that may be formed may be used to deliver drugs or other APIs transdermally to the skin's surface. The liposomes that may be formed using embodiments of the present disclosure may be stabilized by the phospholipids, in addition to their small and relatively uniform particle size. Various molecules from those having a low molecular weight, such as glucose, to those having a high molecular weight, such as peptides and proteins, may be incorporated in liposomes. Water soluble compounds/drugs may be present in aqueous compartments while lipid soluble compounds/drugs and amphiphilic compounds/drugs may insert themselves in phospholipid bilayers. The liposomes having drugs may be administered by various routes, including intravenous, oral inhalation, local application, and ocular, among others. Because of this, liposomes may be used for the treatment of many diseases. Liposomes may be either unilamellar or multilamellar.

Additionally, due to their amphiphilic character, liposomes may be a powerful solubilizing system for a wide range of compounds. In addition to these physico-chemical properties, liposomes may exhibit many special biological characteristics, including specific interactions with biological membranes and various cells. These properties point to several possible applications with liposomes as the solubilizers for difficult-to-dissolve substances, dispersants, sustained release systems, delivery systems for the encapsulated substances, stabilizers, protective agents, microencapsulation systems and micro reactors, among others. Liposomes may be made entirely from naturally occurring substances and may be, therefore, nontoxic, biodegradable, and non-immunogenic.

Another component present in NPE composition described here may be oils that are rich sources of essential fatty acids, behenic acid, and oleic acid. The supply of essential fatty acids and antioxidant molecules may restore the cutaneous permeability and the function of the skin barrier. The supply of essential fatty acids and antioxidant molecules may also contribute to the control of the imperceptible water loss and maintain moisture of the skin.

Behenic acid and oleic acid, when used by themselves, may be irritating when applied to the skin, which makes behenic acid and oleic acid difficult to use as permeation enhancers. While having an irritating effect on the skin, these acids may also be effective vehicles at delivering APIs through the skin. In one embodiment, NPE composition may include pracaxi oil.

Pracaxi Oil

Pracaxi oil may be rich in organic acids with antioxidant, antibacterial, and antifungal properties. Pracaxi oil may be obtained from the seed oil of Pentaclethara macroloba tree. Pracaxi oil may include about 20% w/w behenic acid and about 35% w/w oleic acid. In some cases, it may include more than these percentages. As the behenic acid and oleic acid may be present in the oil, the effects of the acids may be less irritating on the skin, and as such makes the oil a good choice for one of the ingredients of a penetration enhancer. This oil has been widely employed for its cosmetic, therapeutic, and medicinal properties. Scientific studies have shown that pracaxi oil may have strong antibacterial, antiviral, antiseptic, antifungal, anti-parasitic, and anti-hemorrhagic properties.

The oil may have a high amount of solid matter, not fatty acids, which make it solidify in cooler temperatures. The solid matter has gentle moisturizers and high cellular renewal properties, includes Vitamin E and has essential fatty acids, which may make it a suitable oil for products intended to address sensitive skins.

The fatty acid composition of pracaxi oil is illustrated below in table 2.

TABLE 2
Fatty Acid Composition of Pracaxi Oil.

| | Carbon | |
|---|---|---|
| Fatty Acidds | Atoms | Composition % |
| Lauric | 12:00 | 1.3000 |
| Myristic | 14:00 | 1.2100 |
| Palmitic | 16:00 | 2.0400 |
| Stearic | 18:00 | 2.1400 |
| Oleic | 18:10 | 44.3200 |
| Linoleic | 18:20 | 1.9600 |
| Linolenic | 18:30 | 2.3000 |
| Behenic | 22:00 | 19.6700 |
| Lignoceric | 24:00 | 14.8100 |

Plukenetia volubilis Seed Oil

Another oil that may be used in some embodiments in combination with pracaxi oil is Plukenetia volubilis seed oil, also known as Inca Inchi. Plukenetia volubilis seed oil is native to the Amazon Rainforest. The seeds of Inchi may be high in protein (around 27% w/w) and oil (around 35% w/w to around 60% w/w) content. Plukenetia volubilis seed oil extracted from the Plukenetia volubilis plant may be one of the largest plant sources of the Omega family of fatty acids, including a high concentration of protein. Plukenetia volubilis seed oil may also be rich in iodine and vitamin A and vitamin E. Plukenetia volubilis seed oil may be a natural oil with an exceptional content in polyunsaturated fatty acids (greater than 90% w/w) and tocopherols (1.5 to 2 g/kg). Plukenetia volubilis seed oil may be a unique vegetable oil having both essential fatty acids in such a high amount, including 49% w/w of alphalinolenic acid (omega-3) and 34% of linoleic acid (omega-6). While Plukenetia volubilis seed oil has a very high amount of fatty acids, it may also have high amounts of behenic acid (10% w/w to 30% w/w) and oleic acid (35% w/w to 80% w/w).

Inaja Oil

Still yet another oil that may be used is from a tree called Maximiliana maripapalm, or Inaja. Inaja is an indigenous Amazonian palm widespread in the state of Para, growing around the Amazon River estuary. Inaja may have one of the highest sources of lauric acid (greater than 40% w/w) and oleic acid (greater than 15% w/w). Further, the highest concentration of fatty acids found in the Inaja may be found in the kernal oil, as opposed to the pulp oil. Oil from Inaja is extracted from the fruits of the Inaja palm, which may include of about 70% w/w short-chain fatty acids, including lauric acid and myristic acid. This palm has been used in the production of bar soap because of its high concentration of lauric acid. The fatty acid composition of Inaja kernel oil is shown in table 3 below.

TABLE 3
Fatty Acid Composition of Inaja Kernel Oil.

| Fatty | Carbon | Composition |
|---|---|---|
| Acids | Atoms | % |
| Lauric | 12:00 | 40.5000 |
| Myristic | 14:00 | 25.0000 |
| Palmitic | 16:00 | 9.0000 |
| Stearic | 18:00 | 2.4000 |
| Oleic | 18:10 | 10.8000 |
| Linoleic | 18:20 | 1.9600 |
| Linolenic | 18:30 | 2.4000 |
| Behenic | 22:00 | trace |
| Lignoceric | 24:00 | trace |

As mentioned, behenic acid, lauric acid, oleic acid, and other fatty acids, when used by themselves, may be very rough on the skin. But, when an oil such as Plukenetia volubilis seed oil and/or pracaxi oil and/or inaja oil are used, they may work to enhance the restoration of cutaneous barrier organization and epidermal elasticity, in addition to contributing to the control of imperceptible water loss, thus maintaining skin hydration. This may be, at least in part, due to the high amounts of essential fatty acids in these oils. The link between skin permeation and hydration is clear. Increasing the permeability of the stratum corneum may be achieved by the increase of water content in this tissue. Hydration by occlusion may cause a swelling of the corneocytes and, subsequently, may increase the skin permeation of APIs. Here, the utilization of physiological lipids, essential fatty acids, and phospholipids, may provide penetration power with restorative benefits to the skin. While Plukenetia volubilis seed oil, pracaxi oil, and inaja oil have been mentioned here, other oils may also be used in alternative compositions, including patauá oil or seje oil.

Seje Oil

Seje or Patauá oil is extracted from the mesocarp of the patauá palm and generally appears as a greenish-yellow and transparent liquid, with little odor and taste, having the physical appearance and composition of fatty acids that are similar to olive oil (Oleo europaea). It may have high content of unsaturated fatty acids. Due to its high content of oleic acid, seje oil may be used as skin moisturizers. The dry mesocarp of patauá palm may include about 7.4% w/w protein and possess an excellent amino acid composition. Because of this, the protein of patauá may be one of the most valuable found among plants and may be compared with the meat or milk from cattle. The most abundant sterols may be Δ⁵avenosterol and β-sitosterol, with relative contents of about 35% w/w and about 38% w/w, respectively. The most abundant aliphatic alcohols may be those with 7, 8 and 10 carbon atoms. Among tocopherols, α-tocopherol may be predominant. Aldehydes, such as heptanal, octanal, and decanal may be present in the volatile fraction along with terpenoid compounds.

The fatty acid composition of seje oil is illustrated below in table 4.

TABLE 4
Fatty Acid Composition of Seje Oil.

| | Carbon | |
|---|---|---|
| Fatty Acids | Atoms | Composition % |
| Palmitic | 16:00 | 13.2 |
| Polmitolcic | 16:10 | — |
| Stearic | 18:00 | 3.6 |
| Oleic | 18:10 | 77.7 |
| Linoleic | 18:20 | 2.7 |
| Linolenic | 18:30 | 0.6 |
| Archidic | 20:00 | 2 |
| Unsaturated | | 81.6 |

Skin Lipids

Another component of NPE composition may be skin lipids. Examples of skin lipids that may be used in NPE composition may include ceramides and/or squalene. Ceramides are the major lipid constituent of lamellar sheets. Ceramides may be a structurally heterogeneous and complex group of sphingolipids including derivatives of sphingosine bases in amide linkage with a variety of fatty acids. Differences in chain length, type, and extent of hydroxylation and saturation may be responsible for the heterogeneity of the epidermal sphingolipids. Ceramides may play an important role in structuring and maintaining the water permeability barrier function of the skin. In conjunction with the other stratum corneum lipids, they may form ordered structures. A structured semi-occlusive barrier that increases skin hydration may be a positive influence on the penetration of APIs.

Another skin lipid that may be used is squalene, which is a lipid fat in the skin. When used together with a ceramide and a phospholipid, such as phosphatidylcholine, the formulation is mild such that it may be used on even sensitive skin. Squalene may also help to decrease water evaporation, thus speeding up skin permeation of actives and decreasing irritation made by surfactants found in emulsions. Squalene, being a natural emollient, may impart an elegant feel to formulations in which it is used. Squalene may be excellent for use in skin care and to help skin to retain moisture and feel soft and conditioned without feeling greasy.

Butters

Yet another component of NPE composition may be butters rich in linoleic acid and linolenic acid. One example of this type of butter may be Butyrospermum parkii butter, also known as shea butter. Other exemplary butters that may be used in embodiments of the present disclosure may include cupuacu butter, buriti butter, passionfruit butter, mango butter, tucuma butter, palm butter, murumu butter, chamomile butter, cocoa butter, orange butter, lemon grass butter, avocado butter, tamanu butter, aloe butter, shea butter, monoi butter, pomegranate butter, almond butter, jojoba butter, red palm butter, acai butter, olive butter, matcha green tea butter, brazil nut butter, macadamia butter, kokum butter, mafura butter, coffee butter, tucuma butter, ucuúlba butter, bacuri butter, and chamomile butter.

In embodiments of the present disclosure, the use of behenic acid, oleic acid, phospholipids, and the omega family may enhance the permeation of drugs or other active ingredients through the skin in-vitro and in-vivo.

As mentioned, NPE composition may be produced such that the size of the particles may range between about 5 microns and about 20 microns, which may provide a more stable vesicle than if the particle sizes were larger. Various methods may be used to produce particle sizes of about 5 microns to about 20 microns. In one embodiment, a high pressure homogenizer may be used.

While concentrations of the components included in NPE composition described here may vary, table 5 below illustrates exemplary concentrations, including the four main components described above, a concentration range, and optimal concentrations for each of the four components.

TABLE 5
Exemplary Concentrations of Components within NPE Composition.

| Ingredients | Range Concentration (%) | Preferred Concentration (%) |
|---|---|---|
| Phospholipids | 0.05%-5% | 2% |
| Oils | 1%-20% | 3% |
| Skin Lipids | 0.1%-3% | 0.5% |
| Butters | 1%-10% | 2% |

In one embodiment, the formulation may include between about 5% w/w and about 0% w/w of Phosal 75 SA (alcohol; purified phosphatidylcholine; safflower oil; glyceryl stearate; coconut oil, ascorbyl palmitate); between about 5% w/w and about 40% w/w of DMS 3015 (water, alcohol, caprylic/capric triglyceride, hydrogenated lecithin, Butyrospermum parkii butter, squalene, and ceramide 3); between about 5% w/w and about 20% w/w of Inca Inchi (Plukenetia volubilis seed oil, tocopherol); between about 5% w/w and about 40% w/w of pracaxi oil; and between about 10% w/w and about 90% w/w of purified water.

NPE composition may include a combination of about 0.05% w/w to about 5% w/w of one or more phospholipids, about 1% w/w to about 20% w/w of one or more oils having essential fatty acids, such as behenic acid, and oleic acid, where one of the one or more oils may be pracaxi oil, about 0.1% w/w to about 3% w/w of one or more skin lipids, and about 1% w/w to about 10% w/w of a butter having linoleic acid and linolenic acid.

In another embodiment, a composition to be used for skin permeation is provided. The composition may include a combination of a hydrogenated phospholipid, an unsaturated phospholipid, pracaxi oil; Plukenetia volubilis seed oil, ceramide, squalene, and Vitellaria paradoxa (formerly known as Butyrospermum Parkii) butter.

In other embodiments, the composition may include a combination of about 10% w/w to about 50% w/w of pracaxi oil, about 15% w/w to about 40% w/w of patauá oil, about 10% w/w to about 30% w/w of inaja oil, and about 10% w/w to about 30% w/w of one or more suitable emollients. Furthermore, other suitable composition may include a combination of about 1% w/w to about 20% w/w of pracaxi oil, about 10% w/w to about 40% w/w of one or more phospholipids, about 5% w/w to about 20% w/w of one or more of Patauá oil or Inaja oil, and about 5% w/w to about 30% w/w of one or more emulsifiers.

Methods of Elaboration

Methods of Elaboration of NPE Composition

NPE composition may be produced such that the size of the particles may range between about 5 microns and about 20 microns, which may provide a more stable vesicle than if the particle sizes were larger. Various methods may be used to produce disclosed NPE composition. In one embodiment, a homogenizer may be used. Employing a homogenizer, phenoxybenzamine transdermal composition may be put under extreme pressure and forced through very small openings, employing a suitable rotor. NPE composition may be cycled through a number of times to achieve the desired particle size. In one embodiment, a high shear homogenizer may be employed, such as a high shear rotor-stator homogenizer under negative pressure. In one embodiment, an IKA Master Plant homogenizer may be used, which can achieve RPMs over 8,000 RPM.

Methods of Elaboration of Phenoxybenzamine Transdermal Composition

In one embodiment, in order to produce phenoxybenzamine transdermal composition, APIs may be mixed in a first vessel. The mixture within the first vessel may be heated to about 60° C., with slow mixing at a mixing speed of about 500 RPM. The heat may then be stopped and the mixing speed may be increased to 1000 RPM. In a second vessel, NPE composition, whose components are listed above, may be mixed. The contents of the two vessels may be mixed together at a mixing speed of about 5000 RPM for about 2 to about 5 minutes, therefore producing phenoxybenzamine transdermal composition. The mixing may then be stopped such that phenoxybenzamine transdermal composition may be packaged in suitable containers.

Application

In some embodiments, disclosed phenoxybenzamine transdermal composition may be applied manually with or without an applicator such as a swab, brush, cloth, pad, sponge, or with any other suitable applicator, such as a solid support including paper, cardboard, or a laminate material, including material with flocked, glued, or otherwise fixed fibers.

Disclosed phenoxybenzamine transdermal composition, when applied on a body surface, may deliver a therapeutically effective amount of phenoxybenzamine to the systemic circulation of the patient. In particular, transdermal phenoxybenzamine composition may be used to deliver a suitable amount of phenoxybenzamine to achieve a predetermined bloodstream level of phenoxybenzamine, serving as a nerve blocker that may help treating neuropathic pain.

A daily effective amount of the phenoxybenzamine transdermal composition of the disclosure may be provided, for example, in a single dose. The amount per administered dose of phenoxybenzamine transdermal composition, duration, and frequency, may depend on factors such as the nature and severity of the condition, age and general health of the subject, the tolerance of the subject to the phenoxybenzamine transdermal composition, the response of the disease to therapy, and duration and profile of the symptoms experienced by the subject.

Types of neuropathic pain that may be treated with phenoxybenzamine transdermal composition may include fibromyalgia, myofascial pain syndrome, tension headache, temporomandibular joint dysfunction (TMD), neck and low back pain syndromes, migraine headache, sciatica, plantar fasciitis, complex regional pain syndrome, and restless leg syndrome, among others.

### Examples

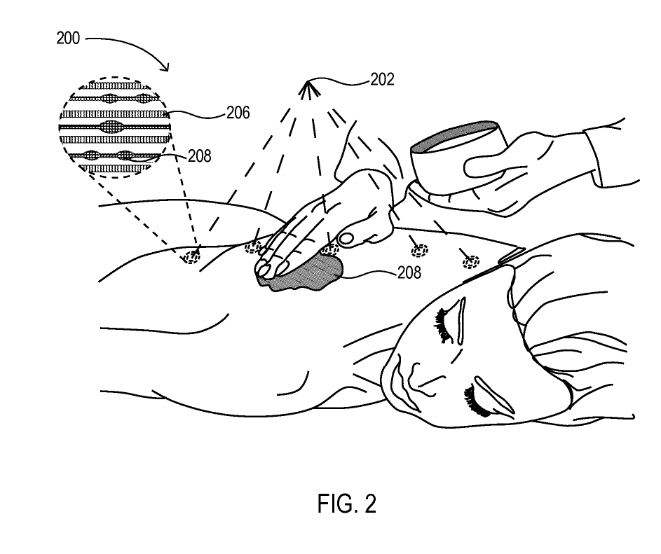

Example #1 is an embodiment of application of phenoxybenzamine transdermal composition for myofascial pain 200, shown in FIG. 2. Accordingly, myofascial trigger points 202 may first be located by a physician using known-in-the art techniques, such as palpating potential myofascial trigger points 202 and subsequently applying needle electromyography (EMG) test; then, phenoxybenzamine transdermal composition 204 may be applied on myofascial trigger points 202, alleviating myofascial pain. Myofascial trigger points 202 include normal fibers 206 and contraction knots 208, the latter of which may produce spontaneous EMG activity that may be blocked by effects of phenoxybenzamine transdermal composition 204.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A method of treating neuropathic pain, comprising applying to the skin an effective amount of a pharmaceutical composition that comprises phenoxybenzamine and at least one permeation enhancement composition.

2. The method according to claim 1, wherein the pharmaceutical composition comprises about 5 mg/g to about 120 mg/g phenoxybenzamine.

3. The method according to claim 1, wherein the pharmaceutical composition comprises about 15 mg/g phenoxybenzamine.

4. The method according to claim 1, wherein the pharmaceutical composition comprises about 20% by weight to about 99.95% by weight of the at least one permeation enhancement composition.

5. The method according to claim 1, wherein the pharmaceutical composition comprises about 50% by weight of the at least one permeation enhancement composition.

6. The method according to claim 1, wherein the pharmaceutical composition is a gel.

7. The method according to claim 1, wherein the pharmaceutical composition is a liquid.

8. The method according to claim 1, wherein the pharmaceutical composition is administered once per day.

9. The method according to claim 1, wherein the pharmaceutical composition is administered in multiple doses per day.

10. The method according to claim 1, wherein the pharmaceutical composition is administered to at least one myofascial trigger point.

11. The method according to claim 1, wherein the at least one permeation enhancement composition comprises one selected from the group comprising a phospholipid, an oil having essential fatty acids, at least one skin lipid, a butter, and combinations thereof.

12. The method according to claim 11, wherein the butter comprises at least one selected from the group consisting of linoleic acid, linolenic acid, and combinations thereof.

13. The method according to claim 11, wherein the essential fatty acids are selected from the group consisting of behenic acid, oleic acid, and combinations thereof.

14. The method according to claim 1, wherein a portion of the at least one permeation enhancement composition has a particle size of about 5 microns to about 20 microns.

15. The method according to claim 1, wherein the treating of neuropathic pain comprises treatment of one selected from the group consisting of fibromyalgia, myofascial pain syndrome, tension headache, temporomandibular joint dysfunction, neck and low back pain syndromes, migraine headache, sciatica, plantar fasciitis, complex regional pain syndrome, restless leg syndrome, and combinations thereof.

16. The method according to claim 1, wherein the at least one permeation enhancement composition comprises about 0% w/w to about 5% w/w of Phosal 75 SA, about 5% w/w to about 40% w/w of DMS 3015, about 5% w/w to about 20% w/w of Inca Inchi, about 5% w/w to about 40% w/w of pracaxi oil, and about 10% w/w to about 90% w/w of water.

17. The method according to claim 1, wherein the at least one permeation enhancement composition comprises about 0.05% w/w to about 5% w/w of one or more phospholipids.

18. The method according to claim 1, wherein the at least one permeation enhancement composition comprises about 1% w/w to about 20% w/w of at least one oil having essential fatty acids.

19. The method according to claim 1, wherein the at least one permeation enhancement composition comprises about 0.1% w/w to about 3% w/w of at least one skin lipid.

20. The method according to claim 1, wherein the at least one permeation enhancement composition comprises about 1% w/w to about 10% w/w of at least one butter.
